SEQUENCE LISTING (24 sequences)

SEQ ID NO: 1 - DNA, 25 bp, Artificial Sequence
  primer
catgaccacagtccatgccatcact

SEQ ID NO: 2 - DNA, 25 bp, Artificial Sequence
  primer
tgaggtccaccaccctgttgctgta

SEQ ID NO: 3 - DNA, 21 bp, Artificial Sequence
  primer
aactggcacacctcaagatgt

SEQ ID NO: 4 - DNA, 21 bp, Artificial Sequence
  primer
tcaagggtattaggcaagggg

SEQ ID NO: 5 - DNA, 21 bp, Artificial Sequence
  primer
tccgccaagccaagcacgaag

SEQ ID NO: 6 - DNA, 21 bp, Artificial Sequence
  primer
catcccgcatctccacagtct

SEQ ID NO: 7 - DNA, 20 bp, Artificial Sequence
  primer
cctttggacacctattcagg

SEQ ID NO: 8 - DNA, 20 bp, Artificial Sequence
  primer
gtgagtgtgtcagctggaag

SEQ ID NO: 9 - DNA, 26 bp, Artificial Sequence
  primer
atgaccatccttttccttactatggt

SEQ ID NO: 10 - DNA, 26 bp, Artificial Sequence
  primer
tcttccccttttaatggtcagtgtac

SEQ ID NO: 11 - DNA, 20 bp, Artificial Sequence
  primer
gtcctcttgctgtccagctc

SEQ ID NO: 12 - DNA, 20 bp, Artificial Sequence
  primer
gaagggcacagtttggagtg

SEQ ID NO: 13 - DNA, 20 bp, Artificial Sequence
  primer
ccaaggacgcagctttctat

SEQ ID NO: 14 - DNA, 20 bp, Artificial Sequence
  primer
ctccggtgagtcctgttgaa

SEQ ID NO: 15 - DNA, 21 bp, Artificial Sequence
  primer
attttattcaagccaccatta

SEQ ID NO: 16 - DNA, 21 bp, Artificial Sequence
  primer
gatacatccacaccgtttagc

SEQ ID NO: 17 - DNA, 18 bp, Artificial Sequence
  primer
aacggcggcttcttcctg

SEQ ID NO: 18 - DNA, 20 bp, Artificial Sequence
  primer
agcagacattggaagaaaca

SEQ ID NO: 19 - DNA, 20 bp, Artificial Sequence
  primer
accagacgatgatgggacag

SEQ ID NO: 20 - DNA, 20 bp, Artificial Sequence
  primer
gccagcacacactcatctat

SEQ ID NO: 21 - DNA, 20 bp, Artificial Sequence
  primer
gaggctgtctcagagcctgt

SEQ ID NO: 22 - DNA, 20 bp, Artificial Sequence
  primer
cttgccgctgtccacttatc

SEQ ID NO: 23 - DNA, 19 bp, Artificial Sequence
  primer
tcagacttccaccgagcag

SEQ ID NO: 24 - DNA, 19 bp, Artificial Sequence
  primer
aggggaaagatcatggccc

(19) United States
(12) Patent Application Publication
(10) Pub. No.: US 2010/0022003 A1
(43) Pub. Date: Jan. 28, 2010

(54) Therapeutic cell medicine comprising skin tissue derived stem cell

(76) Inventor: Soo Kyung Kang, Busan (KR)

Correspondence Address: CHRISTIE, PARKER & HALE, LLP, PO BOX 7068, PASADENA, CA, 91109-7068, US

(21) Appl. No.: 12/311,135

(22) PCT Filed: Sep. 18, 2007
(86) PCT No.: PCT/KR2007/004528

§ 371 Date: Apr. 28, 2009

(30) Foreign Application Priority Data
Sep. 21, 2006 (KR) 10-2006-0091798

(51) Int. Cl. C12N 1/21; C12N 5/06

(52) U.S. Cl. 435/377; 435/325

(57) ABSTRACT

Provided is a cell therapeutic agent for treatment of neurological disorders, comprising skin-derived progenitor cells (SPCs). More specifically, the present invention provides a cell therapeutic agent for treatment of neurological disorders, comprising skin-derived progenitor cells (SPCs) isolated from skin tissues and a method for differentiation of the skin-derived progenitor cells (SPCs) into neural cell lineages. The cell therapeutic agent in accordance with the present invention is therapeutically effective for the treatment of the neurological disorders and diseases such as Parkinson's disease, Alzheimer's disease, Huntington's disease and Amyotrophic Lateral Sclerosis (ALS) caused by neural injury, and neurological deficits due to cerebral apoplexy, ischemia and spinal cord injury. Further, the present invention enables transplantation of autologous cells to thereby minimize adverse side effects.

### TECHNICAL FIELD

The present invention relates to a cell therapeutic agent for treatment of neurological disorders, comprising skin-derived progenitor cells (SPCs). More specifically, the present invention relates to a cell therapeutic agent for treatment of neurological disorders, comprising skin-derived progenitor cells (SPCs) isolated from skin tissues and a method for differentiation of the skin-derived progenitor cells (SPCs) into neural cell lineages.

### BACKGROUND ART

Stem cells refer to immature cells that have a self-renewal capacity and a long-term viability and still retain the potential to differentiate into diverse types of specific specialized cells and tissues. Pluripotent embryonic stem cell lines (ES cell lines) obtained in the embryo development stage have an indefinite differentiation potential, but suffer from various problems that must be resolved in the near future, such as the risk of teratoma formation, provoking of ethical concerns, and immunological problems associated with utilization of the stem cells as a cell therapeutic agent. Fortunately, adult stem cells, which have the performance comparable to that of the embryonic stem cells, were found in various human organs, and mesenchymal stem cells were isolated from mesoderm-derived bone marrow, adipose tissues, umbilical cord blood, and the like, in conjunction with various reports of study and research results. However, the mesenchymal stem cells suffer from a significant disadvantage associated with a very low efficiency of differentiation into neural cells.

In recent years, intensive research and study on treatment of incurable diseases via cell therapy using stem cells has been actively undertaken by domestic and foreign research groups and institutions. Accordingly, there have been reported numerous research results on the utilization of the stem cells in the treatment of various diseases and disorders such as cerebral diseases (e.g. Parkinson's disease and Alzheimer's disease), spinal cord injury (SCI), hepatic cirrhosis, diabetes, myocardial infarction and the like.

Spinal cord injury (SCI) is considered to be a demyelinating central nervous system (CNS) disease. The damaged myelin may be repaired through the induction of proliferation, differentiation and migration of autologous oligo precursor cells or autologous neural stem cells (NSCs). However, such spontaneous processes are not enough to achieve a sufficient recovery of the damaged lesions, and therefore a complete recovery of the disease essentially requires a cell therapeutic process involving external infusion of oligo precursor cells or differentiated myelin-forming neural progenitor cells. Regulation of proliferation, differentiation and migration of neural cells or neural progenitor cells can be effectively carried out by optimization of in vitro cell culture conditions. On the other hand, in order to maximize therapeutic efficiency of the aforementioned method, there has been proposed a combined application of a gene and cell therapy in conjunction with the development and utilization of a vector capable of overexpressing certain cytokines.

Skin-derived progenitor cells (SPCs), in conjunction with neural cell lineages, belong to ectodermal stem cells. SPCs can be isolated and collected from skin tissues of the tissue-injury subjects, and cultivation of such cells is advantageously not complicated and troublesome. In addition, it is known that the stem cells derived and isolated from the skin tissue can be stably multiplied to significant amounts of cells by a cell culture process, and the thus-cultured stromal cells can differentiate into mesodermal tissues as well as ectodermal/endodermal tissues such as liver, nerve cells, and the like. As a therapeutic approach to the treatment of diseases by taking advantage of the infinite differentiation potential of such skin stem cells, it may be possible to appropriately control in vitro culture or differentiation conditions and further it may employ the cells as an important material to construct the cell therapy involving cells and genes in an established animal disease model. As an attempt to treat the diseases using the skin stem cells, Korean Patent Application Publication No. 2006-0016540 A1 discloses a method for isolation of skin stem cells and preparation of artificial skin. However, to the best of our knowledge, there is no case in which application of the stem cells to the treatment of neurological disorders was made via induction of differentiation of the stem cells into neural cells.

### DISCLOSURE OF INVENTION

### Technical Problem

As a result of a variety of extensive and intensive studies and experiments to treat neurological disorders using the aforementioned stem cells, the inventors of the present invention have confirmed that transplantation of skin-derived progenitor cells into a mouse model of spinal cord injury exhibits high neural differentiation potency, thereby leading to efficient recovery from the spinal cord injury. The present invention has been completed based on these findings.

Therefore, the present invention has been made in view of the above problems, and it is an object of the present invention to provide a cell therapeutic agent for treatment of neurological disorders, comprising skin-derived progenitor cells with a high efficiency of differentiation into neural cells.

It is a further object of the present invention to provide a method for differentiation of the aforesaid skin-derived progenitor cells into neural cells.

### Technical Solution

In accordance with an aspect of the present invention, the above and other objects can be accomplished by the provision of a cell therapeutic agent for treatment of neurological disorders, comprising skin-derived progenitor cells isolated from skin tissues.

In accordance with another aspect of the present invention, there is provided a method for differentiation of skin-derived progenitor cells into neural cells, comprising (a) removing subcutaneous fat from skin tissue to obtain skin-derived progenitor cells; and (b) culturing the isolated skin-derived progenitor cells in a neuronal differentiation medium containing at least one differentiation-promoting factor selected from the group consisting of BDNF(Brain-Derived Neurotrophic Factor), bFGF(basic fibroblast growth factor) and combination thereof to thereby achieve differentiation of the skin-derived progenitor cells into neural cells.

In the present invention, the differentiated neural cells are characterized by the expression of one or more genes selected from the group consisting of Nestin, GFAP, TuJ, TrkB, MAP2, NSE, NeuN, BDNF, SDF1, NGF, GDNF, bFGF, EGF, FGFR2 and MBP genes.

In accordance with yet another aspect of the present invention, there is provided neural cells differentiated from the skin-derived progenitor cells by the aforementioned method, and a cell therapeutic agent for treatment of neurological disorders, comprising the neural cells.

In the present invention, the neurological disorder may be selected from the group consisting of Parkinson's disease, Alzheimer's disease, Huntington's disease, Amyotrophic Lateral Sclerosis (ALS) and neurological deficits due to cerebral apoplexy, ischemia and spinal cord injury.

### ADVANTAGEOUS EFFECTS

The present invention provides a cell therapeutic agent for treatment of neurological disorders, comprising skin-derived progenitor cells with a high differentiation into neural cell lineages. The cell therapeutic agent in accordance with the present invention is therapeutically effective for the treatment of the neurological disorders and diseases such as Parkinson's disease, Alzheimer's disease, Huntington's disease and Amyotrophic Lateral Sclerosis (ALS) caused by neural injury, and neurological deficits due to cerebral apoplexy, ischemia and spinal cord injury. Further, the present invention enables transplantation of autologous cells to thereby minimize adverse side effects.

### BRIEF DESCRIPTION OF THE DRAWINGS

FIG. 1 is a graph showing changes in cell growth time versus passages of skin-derived progenitor cells in accordance with the present invention;

FIG. 2 is a photograph confirming a differentiation capacity of skin-derived progenitor cells in accordance with the present invention. 2A: Confirmation of adipogenic differentiation. 2B: Confirmation of osteogenic differentiation via Von Kossa staining. 2C: Confirmation of chondrogenic differentiation via Safranin O staining;

FIG. 3 is a photograph confirming a differentiation capacity of skin-derived progenitor cells in accordance with the present invention into neural cells by an immunochemical method;

FIG. 4 is a photograph confirming a differentiation capacity of skin-derived progenitor cells in accordance with the present invention into neural cells by RT-PCR;

FIG. 5 is a graph showing a recovery degree of animals from spinal cord injury (SCI), in a SCI mouse model with transplantation of skin-derived progenitor cells in accordance with the present invention. 5A: Graph showing BBB scores, and 5B: Bar graph showing volumetric changes in spinal cord lesions;

FIG. 6 is a photograph showing histological examination results for tissues of a spinal cord injury (SCI) mouse model with transplantation of skin-derived progenitor cells in accordance with the present invention. 6A: EM(electron microscope) of tissues, and 6B: H & E staining of tissues; and

FIG. 7 is a photograph showing immunochemical examination results for tissues of a spinal cord injury (SCI) mouse model with transplantation of skin-derived progenitor cells in accordance with the present invention.

### BEST MODE FOR CARRYING OUT THE INVENTION

Hereinafter, the present invention will be described in more detail.

In the present invention, the differentiation of skin-derived progenitor cells into neural cells or neural progenitor cells was confirmed by the expression of a gene encoding Nestin, GFAP, TuJ, TrkB, MAP2, NSE, NeuN, BDNF, SDF1, NGF, GDNF, bFGF, EGF, FGFR2 or MBP, which is a neural differentiation marker, or by the production of the aforementioned proteins.

As used herein, the term “skin-derived progenitor cell (SPC)” refers to an autologous skin-derived progenitor cell which is isolated from animal skin and is capable of differentiating into a variety of tissue cell lineages, such as adipocytes, osteoblasts, chondrocytes, neurocytes and the like.

As used herein, the term “oligo cell” is a subgroup of cells belonging to neurogenic lineages (such as astrocytes, neurons, and oligodendrocytes) and refers to a precursor cell which is supposed to form myelin surrounding nerve cell fibers. In the case of a spinal cord disease which is a disease occurring due to apoptosis of motor neurons arising from destruction or loss of the myelin sheath (demyelination), induction of remyelination using the oligo cells is very important for repair of spinal cord injury.

The inventors of the present invention have closely examined cultural and growth characteristics of cultured skin-derived progenitor cells via isolation and culture of mouse skin-derived progenitor cells (mSPCs), and established conditions for efficient differentiation of the skin-derived progenitor cells into ectodermal nerve cells.

In order to evaluate therapeutic effects of the cell therapy on the treatment of demyelinating diseases via the use of skin stem cell-derived neural progenitor cells which were efficiently differentiated into the oligo precursor cells by means of the thus-established cell differentiation conditions and culture method, a mouse model of spinal cord injury was established.

Transplantation of the skin-derived progenitor cells into the thus-established spinal cord injury animal model may be carried out by a conventional cell transplantation technique known in the art. Preferably, there may be employed intravenous injection and intraspinal injection. More preferred is intravenous injection.

According to the results of cell transplantation and therapeutic treatment obtained in the mouse model of spinal cord injury, the cell transplantation resulted in a significant symptomatic improvement and differentiation of considerable numbers of transplanted cells into mature neural cells (neurons and myelin). Further, as another function of the engrafted skin-derived progenitor cells, it is considered that they secrete a substance playing a very important role in migration of adjacent spinal cord-derived neural stem cells into the spinal cord lesions, which consequently leads to migration and differentiation of the spinal cord-derived neural stem cells and further efficient induction of the functional maturation of the neural stem cells into mature cells having neuronal functions, thereby making a great contribution to a functional recovery from spinal cord injuries/disorders.

The cell therapeutic agent of the present invention may be prepared into conventional formulations of cell therapeutic agents known in the art. For example, the cell therapeutic agent may be formulated into an injectable preparation, and may be directly transplanted into spinal cord lesions via a surgical route or otherwise may be intravenously administered and then migrated to the nerve injury sites. The dosage of the skin-derived progenitor cells or the differentiated neural cells contained in the composition of the present invention may vary depending upon type of disease, administration route, age and sex of patient, and severity of disease. Preferably, the composition of the present invention is administered to give a concentration of 10⁴ to 10⁸ cells for the average adult.

On the other hand, BDNF and bFGF, the differentiation-promoting factors used in the method for differentiation of skin-derived progenitor cells into neural cells in accordance with the present invention, are peptidic materials that serve as a growth factor or nutritional factor, and are also growth factors or functional factors that are added to the cell culture medium or differentiation medium which is desired to induce differentiation into neural progenitor cells or neural cells. The aforesaid compound is preferably used in a final working concentration of 10 to 20 ng/mL. In addition to BDNF and bFGF, the neuronal differentiation medium used in the present invention may further contain B27 as a serum-free supplement.

### MODE FOR THE INVENTION

### Examples

Now, the present invention will be described in more detail with reference to the following Examples. These examples are provided only for illustrating the present invention and should not be construed as limiting the scope and spirit of the present invention.

Although the following Examples confirm therapeutic effects of the stem cells on the treatment of spinal cord injury by direct transplantation of skin-derived progenitor cells to a mouse model of spinal cord injury, it will be apparent to those skilled in the art that similar therapeutic effects can also be achieved even with transplantation of skin-derived progenitor cells to the spinal cord injury mouse model after differentiation of the skin-derived progenitor cells into neural cells or neuroprogenitor cells.

### Example 1

### Isolation and Culture of Progenitor Cells from Autologous Skin Tissues

In order to isolate skin-derived progenitor cells from mice, skin tissues were excised from dorsal and ventral epidermis skin of 6-week-old ICR mice (Hyochang Science, Korea). Subcutaneous fat was removed from the thus-obtained skin tissues which were then washed with phosphate buffered saline (PBS).

0.075% collagenase was added to the subcutaneous fat-removed skin tissues, and the resulting mixture was reacted at 37° C. for 30 min to isolate cells from the skin tissues.

α-Dulbecco's Modified Eagle's Medium (DMEM) containing 10% FBS (fetal bovine serum) (GIBCO, USA) was added to the cell fluid that completed the reaction, followed by centrifugation. The stromal cell layer was suspended in DMEM containing 10% FBS and cultured for 48 to 72 hours until the primary culture cells reach a cell density of 70 to 80%. Then, passage culture was carried out using a 0.025% trypsin solution, thereby obtaining skin-derived progenitor cells. The following Examples employed the skin-derived progenitor cells after 5 to 6 passages of the cells.

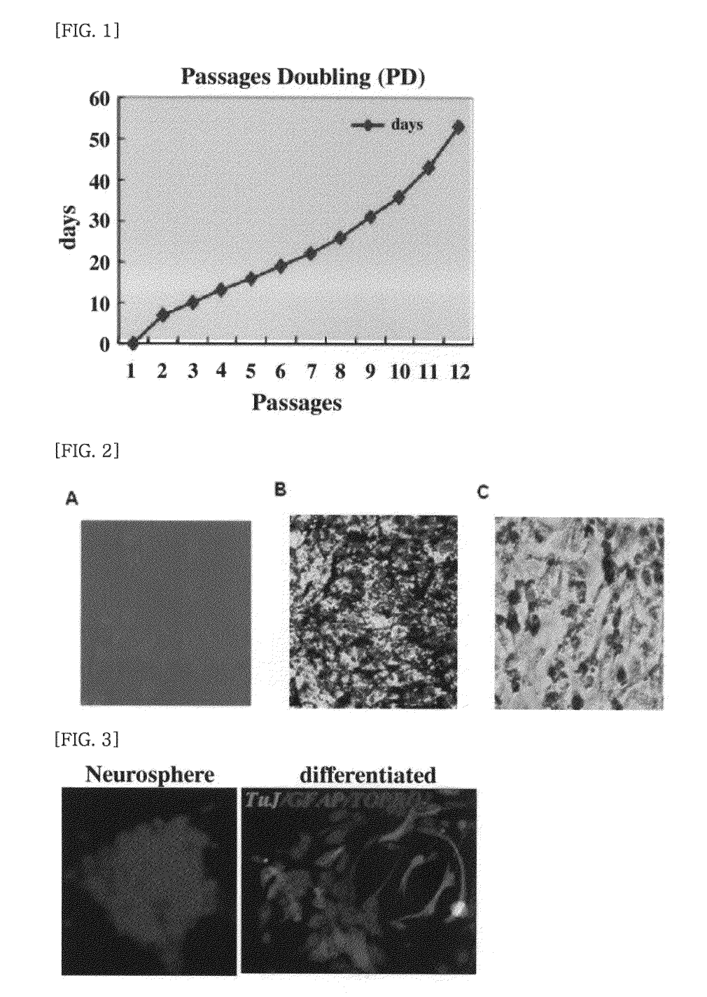

FIG. 1 is a graph showing changes in PD (passage doubling) time versus number of times of passages. As shown in FIG. 1, it can be seen that the skin-derived progenitor cells, isolated by the aforementioned method, can be stably multiplied and obtained in significant numbers of cells by a cell culture process.

### Example 2

### Differentiation Capability of Skin-Derived Progenitor Cells into Mesodermal Cell Lineages

In order to investigate whether the skin-derived progenitor cells obtained in Example 1 are capable of differentiating into mesodermal cell lineages such as adipocytes, osteocytes and chondrocytes, the skin-derived progenitor cells were cultured to differentiate into a specific cell type in a differentiation medium that induces tissue-specific differentiation of cells.

In order to ascertain the adipogenic differentiation capacity of the skin-derived progenitor cells, the cells were cultured to differentiate into adipocytes in an adipogenic differentiation medium containing adipogenic factors, e.g. insulin (Sigma, St Louis, Mo., USA) and dexamethasone (Sigma, St Louis, Mo., USA). In order to confirm the osteogenic differentiation capacity of the skin-derived progenitor cells, the cells were cultured to differentiate into osteocytes in an osteogenic differentiation medium containing osteogenic factors, e.g. glycerophosphate (Sigma, St Louis, Mo., USA), ascorbic acid (Sigma, St Louis, Mo., USA), and dexamethasone (Sigma, St Louis, Mo., USA). Finally, in order to confirm the chondrogenic differentiation capacity of the skin-derived progenitor cells, cells were cultured to differentiate into chondrocytes in a chondrogenic differentiation medium containing chondrogenic factors, e.g. insulin, TGF-beta (Sigma, St Louis, Mo., USA), and ascorbic acid (Sigma, St Louis, Mo., USA).

The degree of adipogenic differentiation was confirmed by microscopic examination of intracellular lipid droplets produced in adipocytes (FIG. 2A). The degree of osteogenic differentiation was confirmed by the formation of calcium-deposited bone nodules via Von Kossa staining (FIG. 2B), whereas the degree of chondrogenic differentiation was confirmed by the presence of Safranin O-stained glycosaminoglycans (FIG. 2C).

As a result, it was confirmed that the skin-derived progenitor cells isolated in Example 1 are capable of differentiating into mesodermal lineages such as adipose tissue, cartilage and bone.

### Example 3

### In Vitro Neurogenic Differentiation of Skin-Derived Progenitor Cells into Neural Cells

In order to use the skin-derived progenitor cells of Example 1 as a cell therapeutic agent for the treatment of spinal cord injury (SCI) which is one of demyelinating diseases, the differentiation capacity of the skin-derived progenitor cells into neural cell lineages was examined.

To induce formation of neurospheres of cells, the skin-derived progenitor cells were suspension-cultured in the neuronal differentiation medium consisting of a Neurobasal medium (Gibco, Rockville, Md., USA) supplemented with B27 (Gibco, USA), 10 μg/mL of BDNF (Brain-Derived Neurotrophic Factor, Sigma, St Louis, Mo., USA) and 20 μg/mL of bFGF (basic fibroblast growth factor, Sigma, St Louis, Mo., USA) for 4 days. The thus-formed neurospheres were spread on substrate-coated culture dishes, and the differentiation efficiency of the neurospheres into the oligo cells was examined.

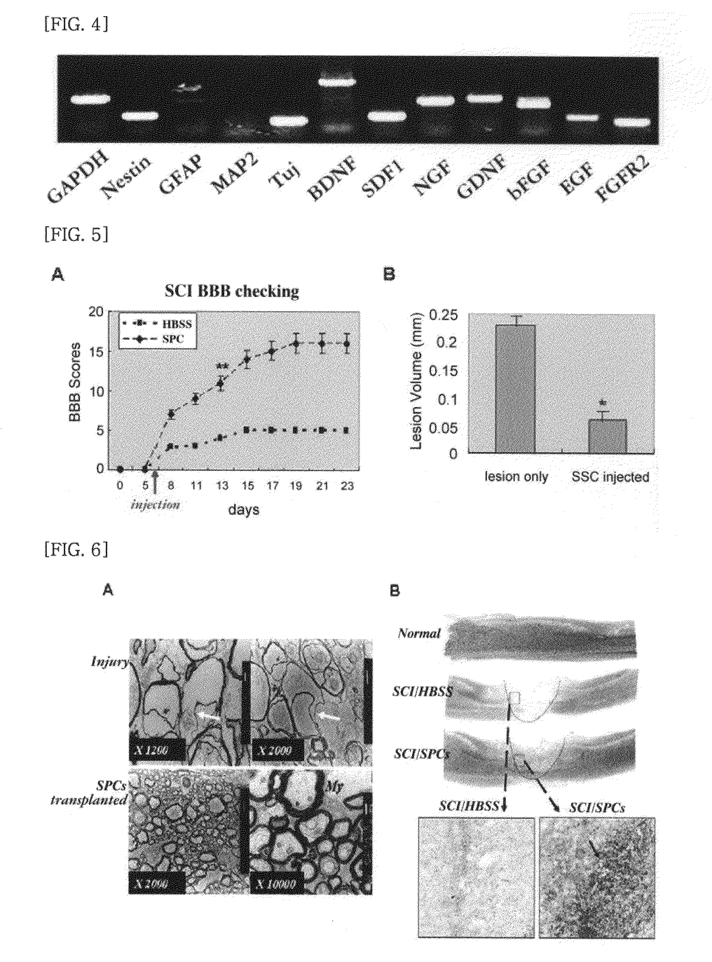

The evaluation of differentiation efficiency of the neurospheres into the oligo cells was optimized by analysis of a relative efficiency via quantitation and qualification of specifically expressed transcripts and proteins, employing real time RT-PCR and an immunochemical method using a specific antibody (FIGS. 3 and 4).

Primer sequences used in analysis of transcripts are set forth in Table 1 below.

TABLE 1

| | Forward Sequence | Reverse Sequence |
|---|---|---|
| Genes | (5′-3′) | (5′-3′) |
| GAPDH | CATGACCACAGTCCATGCCA | TGAGGTCCACCACCCTGTTGCT |
| | TCACT(SEQ ID NO: 1) | GTA(SEQ ID NO: 2) |
| Nestin | AACTGGCACACCTCAAGATG | TCAAGGGTATTAGGCAAGGGG |
| | T(SEQ ID NO: 3) | (SEQ ID NO: 4) |
| GFAP | TCCGCCAAGCCAAGCACGAA | CATCCCGCATCTCCACAGTCT |
| | G(SEQ ID NO: 5) | (SEQ ID NO: 6) |
| Tuj | CCTTTGGACACCTATTCAGG | GTGAGTGTGTCAGCTGGAAG |
| | (SEQ ID NO: 7) | (SEQ ID NO: 8) |
| BDNF | ATGACCATCCTTTTCCTTACT | TCTTCCCCTTTTAATGGTCAGT |
| | ATGGT(SEQ ID NO: 9) | GTAC(SEQ ID NO: 10) |
| SDF-1 | GTCCTCTTGCTGTCCAGCTC | GAAGGGCACAGTTTGGAGTG |
| | (SEQ ID NO: 11) | (SEQ ID NO: 12) |
| NGF | CCAAGGACGCAGCTTTCTAT | CTCCGGTGAGTCCTGTTGAA |
| | (SEQ ID NO: 13) | (SEQ ID NO: 14) |
| GDNF | ATTTTATTCAAGCCACCATTA | GATACATCCACACCGTTTAGC |
| | (SEQ ID NO: 15) | (SEQ ID NO: 16) |
| bFGF | AACGGCGGCTTCTTCCTG | AGCAGACATTGGAAGAAACA |
| | (SEQ ID NO: 17) | (SEQ ID NO: 18) |
| EGF | ACCAGACGATGATGGGACAG | GCCAGCACACACTCATCTAT |
| | (SEQ ID NO: 19) | (SEQ ID NO: 20) |
| FGFR2 | GAGGCTGTCTCAGAGCCTGT | CTTGCCGCTGTCCACTTATC |
| | (SEQ ID NO: 21) | (SEQ ID NO: 22) |
| MAP2 | TCAGACTTCCACCGAGCAG | AGGGGAAAGATCATGGCCC |
| | (SEQ ID NO: 23) | (SEQ ID NO: 24) |

As primary antibodies for immunochemical analysis of the differentiated neural cells, TuJ (Sigma, USA) and GFAP (DAKO, USA) were employed.

As a result, the neurospheres induced from skin-derived progenitor cells were found to express neuron-specific genes. It was also found that the skin-derived progenitor cells form neurospheres, similar to neuroprogenitor cells. Further, it could be seen via the immunochemical method that such cells also express TUJ and MAP2ab at high levels in vivo and in vitro, thus showing high neurogenic differentiation efficiency. In addition, the immunohistochemical analysis results of spinal cord lesions revealed that the skin-derived progenitor cells have an ability to differentiate and mature into myelin expressing MBP.

### Example 4

### Establishment of Spinal Cord Injury Animal Model Using Mice

In order to examine whether the neural cells, which were constructed in Example 3 and were differentiation-induced from the skin-derived progenitor cells, are capable of functionally restoring the impaired neural tissues in myelin- and motor neuron-destroying diseases, a mouse model of spinal cord injury was established.

Establishment of the spinal cord injury animal model using X-ray irradiation and EB injection is evaluated to be a method capable of minimizing the possibility of self-injury recovery.

For this purpose of establishment of the spinal cord injury animal model, 6-week-old adult ICR female mice with induction of the motor neuron- and myelin-destroying diseases were subjected to X-ray irradiation and then administration of ethidium bromide (EB) (Kocsis et al., J. Neuroscience, 1996). X-rays were irradiated at a dose of 40 Grays to animals, and 3 days later the spinous process of T10 was incised, followed by direct injection of 0.5 μl of EB at a concentration of 0.3 mg/mL, or otherwise physical trauma was induced in the T10 region of the spinal cord, thereby leading to destruction of myelin and motor neurons.

The degree of demyelination was indirectly assessed by daily checking the body weight loss and BBB scores including clinical scores of various items (see Table 2 below) in mice with destruction of motor neurons.

In order to avoid lowering of a survival rate of the engrafted cells which may occur due to an overreaction of the immune response appearing after lesion induction, the above-established mouse model received transplantation of progenitor stem cells 3 days after induction of spinal cord injury.

TABLE 2
Behavioral assessment (BBB scores)

| Rat- | |
|---|---|
| ing | Description of Locomotion |
| 0 | No observable hindlimb (HL) movement |
| 1 | Slight movement of one or two joints (usually hip and/or knee) |
| 3 | Extensive movement of two joints (ankle and knee) |
| 5 | Slight movement of two joints and extensive movement of the |
| | third (femoral region) |
| 8 | Sweeping with no weight support or Plantar placement of the |
| | paw with no weight support |
| 10 | Occasional weight supported plantar steps, no FL-HL coordination |
| 12 | Frequent to consistent weight supported plantar steps and |
| | occasional FL-HL coordination |
| 14 | Consistent weight supported plantar steps, consistent FL-HL |
| | coordination |
| 15 | Occasional toe clearance during forward limb advancement |
| 16 | Toe clearance occurs frequently during forward limb advancement |
| 19 | Partial functional recovery of impaired caudal nerves |
| 21 | Complete functional recovery of impaired caudal nerves |

### Example 5

### Transplantation of Skin-Derived Progenitor Cells into Mouse Model of Spinal Cord Injury

In order to transplant the skin-derived progenitor cells isolated in Example 1 into the spinal cord injury mouse model established in Example 4, the cells were labeled with a fluorescent dye carboxyfluorescein diacetate (CFDA).

The skin-derived progenitor cells labeled with the aforementioned fluorescent marker were transplanted into a tail vein of the spinal cord injury mouse model. Cell trans-plantation was carried out by intravenous injection of the cells (at a density of 1×10⁶ cells) into the tail vein of animals, using a syringe with 25-gauge needle. In order to examine therapeutic efficiency following transplantation of the cells, the body weight and clinical scores or BBB scores (0 to 21 points) of the mouse model were daily and periodically measured for a given period of time. For confirmation of functional recovery by comparative analysis between each group, the thus-obtained results were compared with those of normal mice and non-transplanted mice model. 4 weeks after transplantation of the cells, the mouse model with transplantation of the skin-derived progenitor cells exhibited significant symptomatic amelioration corresponding to a 17 point score, based on a total BBB score of 21 points, thus representing that the condition of the animals having paralyzed lower extremities was improved such that the animal can freely step using hindlimb. On the other hand, it can be seen that a negative control group with injection of a buffer solution into the mouse model exhibited no improvements of the disease (FIG. 5A).

Further, as compared to the control group, the mouse model with transplantation of the skin-derived progenitor cells was found to exhibit a significant decrease in a size of the spinal cord lesion after transplantation of the cells (FIG. 5B).

### Example 6

### Histological Analysis of Mouse Model with Transplantation of Skin-Derived Progenitor Cells

Histological analysis was carried out on brain, spinal cord and other organs of mice with transplantation of the skin-derived progenitor cells.

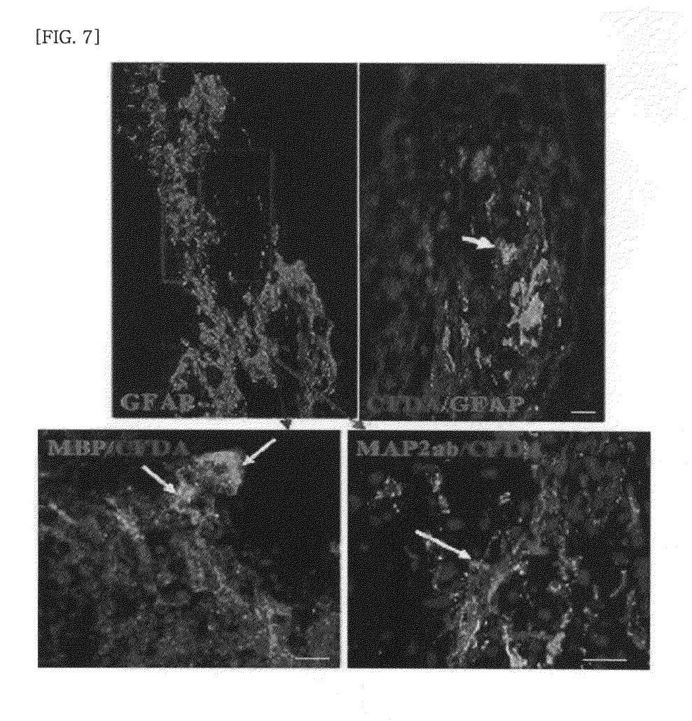

For each animal group of a mouse model with transplantation of the skin-derived progenitor cells, a mouse model with no transplantation of the cells, and a normal mouse group, the brain, spinal cord and other organs were removed and fixed with 4% paraformaldehyde, and then cut into a given thickness of 5 μm. The tissue sections were examined and compared under an electron microscope. Further, the each tissue was embedded in paraffin, cut into a given size, and stained with Hematoxylin & Eosin. Thereafter, destruction of the myelin, reduction of demyelination, and a recovery from demyelination were examined (FIG. 6).

Further, in order to investigate a differentiation degree of the transplanted cells, the cells were labeled with specific antibodies for certain mature cell types, such as GFAP (DAKO, USA), MAP2ab (Sigma, USA), MBP (Sigma, USA) and CFDA (Molecular Probe, USA), using the immunochemical method, and examined under a Confocal Laser Scanning Microscope (FIG. 7).

For comparative quantification of the expression of specific proteins related to certain neural tissues, total RNA was extracted from each tissue using Trizol, and subjected to purification and real time RT-PCR for comparative analysis.

As a result, the cells transplanted through the tail vein migrated via the blood stream to the spinal cord lesions within a short period of time. That is, a very high percent of the injected cells, e.g. about 45%, migrated to the lesion sites. Further, the injected cells were also found in other organs such as brain, lung, kidney and liver. Most of the transplanted cells which migrated to the spinal cord lesions were localized in the cavity of the spinal cord lesions, thus making a contribution to the formation of scar, and the cells were also found in the surrounding tissues adjacent to the lesion sites. As shown in FIG. 6, the damaged surrounding tissues exhibited a relatively high differentiation capacity of the engrafted cells into the neural cells or oligo cells. Upon considering these results, it is believed that advantageous and beneficial effects on the functional recovery of spinal cord injury are obtained by the effects according to the differentiation of skin-derived progenitor cells into neural cells, as well as by the indirect function owing to secretion of growth factors from the skin-derived progenitor cells.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

The cell therapeutic agent in accordance with the present invention is therapeutically effective for the treatment of the neurological disorders and diseases such as Parkinson's disease, Alzheimer's disease, Huntington's disease and Amyotrophic Lateral Sclerosis (ALS) caused by neural injury, and neurological deficits due to cerebral apoplexy, ischemia and spinal cord injury. Further, the present invention enables transplantation of autologous cells to thereby minimize adverse side effects.

1. A cell therapeutic agent for treatment of neurological disorders, comprising skin-derived progenitor cells isolated from skin tissues.

2. The cell therapeutic agent according to claim 1, wherein the neurological disorder is selected from the group consisting of Parkinson's disease, Alzheimer's disease, Huntington's disease, Amyotrophic Lateral Sclerosis (ALS) and neurological deficits due to cerebral apoplexy, ischemia and spinal cord injury.

3. A method for differentiation of skin-derived progenitor cells into neural cells or neural progenitor cells, comprising:
(a) removing subcutaneous fat from skin tissue to obtain skin-derived progenitor cells;
(b) culturing the isolated skin-derived progenitor cells in a neuronal differentiation medium containing at least one differentiation-promoting factor to form neurospheres, wherein the differentiation-promoting factor is selected from the group consisting of BDNF, bFGF and combination thereof; and
(c) culturing the neurospheres to differentiate into neural cells or neural progenitor cells.

4. The method according to claim 3, wherein the differentiated neural cells or neural progenitor cells express one or more genes selected from the group consisting of Nestin, GFAP, TuJ, TrkB, MAP2, NSE, NeuN, BDNF, SDF1, NGF, GDNF, bFGF, EGF, FGFR2 and MBP genes.

5. Neural cells or neural progenitor cells differentiated from the skin-derived progenitor cells by the method of claim 3.

6. A cell therapeutic agent for treatment of neurological disorders, comprising the neuronal cells or neural progenitor cells of claim 5.

7. The cell therapeutic agent according to claim 6, wherein the neurological disorder is selected from the group consisting of Parkinson's disease, Alzheimer's disease, Huntington's disease, Amyotrophic Lateral Sclerosis (ALS) and neurological deficits due to cerebral apoplexy, ischemia and spinal cord injury.
